(19) United States
(12) Plant Patent Application Publication
(10) Pub. No.: US 2016/0345482 P1
(43) Pub. Date: Nov. 24, 2016

(54) Dianthus plant named 'KLEDP15180'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE); Lena Gutbrodt, Bempfingen (DE)

(73) Assignee: Klemm+Sohn GmbH & Co. KG, Stuttgart (DE)

(21) Appl. No.: 14/545,536

(22) Filed: May 18, 2015

(51) Int. Cl. A01H 5/00

(52) U.S. Cl. PLT/276

(57) ABSTRACT

A new dianthus plant particularly distinguished by having light pink flowers with a purple star, good branching, and medium vigor, is disclosed.

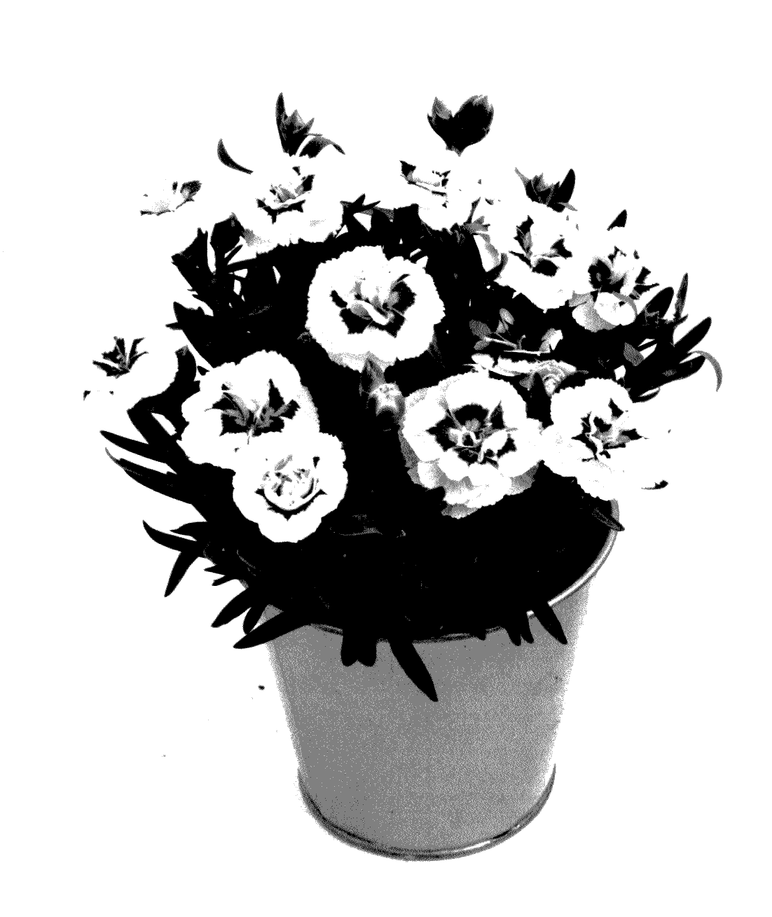

### GENUS AND SPECIES

Dianthus caryophyllus

### VARIETY DENOMINATION

‘KLEDP15180’

### BACKGROUND OF THE NEW PLANT

The present invention comprises a new and distinct variety of dianthus, botanically known as Dianthus caryophyllus and hereinafter referred to by the variety name ‘KLEDP15180’. ‘KLEDP15180’ resulted from a whole plant mutation of ‘Oscar Pink and Purple’ (unpatented) identified in March 2010 in Stuttgart, Germany.

In July of 2010 ‘KLEDP15180’ was first vegetatively propagated by vegetative cuttings. ‘KLEDP15180’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. Light pink flowers with a purple star;

2. Good branching; and

3. Medium vigor.

### CROSS REFERENCE TO RELATED APPLICATIONS

Plant Breeder's Rights for ‘KLEDP15180’ were applied for in Canada on Apr. 2, 2015 (Application number 15-8575). ‘KLEDP15180’ has not been made publicly available or sold more than one year prior to the filing date of this application.

### DESCRIPTION OF THE PHOTOGRAPH

This new Dianthus plant is illustrated by the accompanying photograph which shows an overall view of the plant. The colors shown are as true as can be reasonably obtained by conventional photographic procedures. The photograph is of a plant that is about 3 months-old grown in Stuttgart, Germany in May 2014.

### DESCRIPTION OF THE NEW VARIETY

The following detailed descriptions set forth the distinctive characteristics of ‘KLEDP15180’. The data which define these characteristics were collected from asexual reproductions carried out in Stuttgart, Germany. Data was collected on 22 week-old plants grown in a greenhouse in Stuttgart, Germany in April 2015. Color references are to The R.H.S. Colour Chart of The Royal Horticultural Society of London (R.H.S.), ^{5th} edition (2007).
Classification:
   Family.—Caryophyllaceae.
   Species.—Dianthus caryophyllus.
   Common name.—Pot carnation.
   Variety.—‘KLEDP15180’.
Parentage: Mutation of ‘Oscar Pink and Purple’ (unpatented)
Propagation:
   Type.—Vegetative cuttings.
   Time to produce a rooted young plant.—14 to 16 days.
   Root description.—White, fine.
   Rooting habit.—Lanceolate.
Plant description:
   Plant form.—Potted plant, upright.
   Plant height.—13.0 cm to 15.0 cm.
   Plant diameter or spread.—16.0 cm to 20.0 cm.
Lateral branches:
   Length.—8.0 cm to 10.0 cm.
   Diameter.—0.3 cm to 0.5 cm.
   Internode length.—2.0 cm to 3.0 cm.
   Node length.—0.3 cm to 0.4 cm.
   Node diameter.—0.4 cm.
   Texture.—Smooth.
   Color.—RHS 138A.
Foliage description:
   Arrangement.—Opposite.
   Length.—4.0 cm to 6.0 cm.
   Width.—0.6 cm to 0.8 cm.
   Shape.—Lanceolate.
   Apex.—Acute.
   Base.—Obtuse.
   Margin.—Entire.
   Texture (both surfaces).—Smooth.
   Venation pattern.—Palmate.
   Color.—Immature leaves: Upper surface: RHS 137B Lower surface: RHS 138A Mature leaves: Upper surfaces: RHS 139A Lower surface: RHS 137A Venation: RHS 139A.
Flower description:
   Flower type and habit.—Round, double, small sprays.
   Fragrance.—Present.
   Natural flowering season.—Spring to fall.
   Flower longevity.—10 to 14 days.
   Flower buds.—Length: 1.5 cm to 2.0 cm Diameter: 0.8 cm to 1.0 cm Shape: Elliptic Color: RHS 138A and RHS 143B.
   Corolla.—Quantity of petals/arrangement: 18 to 22, arranged in several whorls Corolla height: 1.5 cm to 2.0 cm Petal length: 3.0 cm Petal width: 1.5 cm to 2.0 cm Petal shape: Spoon shaped Petal apex: Obtuse Petal base: Acute Petal margin: Crenate Petal texture (both surfaces): Smooth Petal color immature and mature: Upper surface: RHS 60A and RHS 62C Lower surface: RHS 65D Calyx: Quantity of sepals/arrangement: 4 to 5, fused Calyx height: 1.8 cm to 2.0 cm Calyx diameter, at the apex: 0.8 cm to 1.0 cm Sepal texture (both surfaces): Smooth Sepal color: Upper surface: RHS 138B and RHS 143A Lower surface: RHS 144B.
Peduncles:
   Length.—10.0 cm to 14.0 cm.
   Diameter.—0.2 cm.
   Strength.—Moderate.
   Texture.—Smooth.
   Color.—RHS N137B.
Reproductive organs:
   Stamens.—Quantity: Moderate Filament length: 0.4 cm to 0.6 cm Filament color: RHS 155B Anther length: 0.1 cm Anther diameter: 0.05 cm Anther color: RHS 19B Pollen amount: Sparse.
   Pistils.—Quantity: 1 Pistil length: 0.1 cm Style length: 1.6 cm to 1.8 cm Style color: RHS 59A Stigma shape: Filamentous Stigma color: RHS 59A Ovary size: 0.4 cm to 0.6 cm Ovary shape: Oval Ovary color: RHS 145A.
Fruit and seed: None observed
Disease and insect/pest resistance: No special observations were made

### COMPARISON WITH PARENTAL LINES AND KNOWN VARIETY

‘KLEDP15180’ is a distinct variety of dianthus. ‘KLEDP15180’ is distinguished from its parent as described in Table 1:

TABLE 1
Comparison with Parental Line

| | Variety | Parent |
|---|---|---|
| Trait | ‘KLEDP15180’ | ‘Oscar Pink and Purple’ |
| Flower color | Light pink with purple star | Pink with purple eye |

‘KLEDP15180’ is most similar to the commercial dianthus plant named ‘Oscar’ also known as ‘KLEDP07089’ (unpatented). Differences between the two varieties are described in Table 2:

TABLE 2
Comparison with Similar Variety

| | Variety | Commercial line |
|---|---|---|
| Trait | ‘KLEDP15180’ | ‘Oscar’ |
| Flower color | Light pink with purple star | Pink with purple eye |

We claim:

1. A new and distinct variety of dianthus plant named ‘KLEDP15180’ as illustrated and described herein.
